(19) United States
(12) Patent Application Publication
(10) Pub. No.: US 2006/0079481 A1
(43) Pub. Date: Apr. 13, 2006

(54) Method of treating/preventing mucositis

(76) Inventor: Rolf Engstad, Tromso (NO)

Correspondence Address: LADAS & PARRY LLP, 224 SOUTH MICHIGAN AVENUE, SUITE 1600, CHICAGO, IL, 60604, US

(21) Appl. No.: 10/962,314

(22) Filed: Oct. 8, 2004

(51) Int. Cl. A61K 31/716

(52) U.S. Cl. 514/54; 536/123.12

(57) ABSTRACT

A method of treating and/or preventing mucositis which is an inflammation and ulceration of the mucous membranes, typically resulting from a toxic reaction to chemotherapy and/or radiotherapy by the application of water soluble immunomodulatory β-glucan.

### FIELD OF THE INVENTION

The present invention relates to methods of treating and/or preventing mucositis resulting from radio- and/or chemotherapy, as well as a glucan for use in a therapeutic composition for treatment and/or prevention of mucositis, as well as uses thereof.

### BACKGROUND OF THE INVENTION

Mucositis is defined as inflammation and ulceration of the mucous membranes, and is a common dose-limiting toxic reaction to chemotherapy and radiotherapy. The definition of mucositis is often restricted to the oropharynx and lips, because of the easy access of these areas for evaluation. However, chemotherapy affects all mucous membranes along the gastrointestinal tract as mitotically active cells are sensitive to this treatment. Complications of mucositis may include fibrosis of salivary glands, muscles and blood vessels, loss of the sense of taste and, in extreme cases, osteoradionecrosis (ORN) of underlying bone.

In RTOG (Radiation Treatment Oncology Group) studies it has been demonstrated a 25% incidence, approximately, of grade 3 and 4 mucositis after radiation therapy delivered in standard conventional dose fractions. Other study groups have reported grade 3 mucositis rates approaching 50%. Moderately accentuated regimens, such as concomitant boost or hyperfractionation, seem to double the incidence of high-grade mucositis, up to 50% to 60%. More aggressive treatment schedules have produced even higher incidences of mucositis; grade 3 from 66% to 86%, and grade 4 from 7% to 48% (Trotti 2000). The prevalence of chemotherapy-induced oral mucositis has generally been reported as ranging from 30% to 39%. A prevalence of 75% has been reported with 5-fluorouracil (Dodd et al. 1996).

As immune compromised mucous membranes represent a potential opening for local and systemic infections and subsequent complications, mucositis is a serious adverse reaction that might lead to reductions or delays in chemotherapy or radiation treatment, consequently having an adverse impact on the curative potential of the primary care. Furthermore, mucositis is very painful and may prevent the patient from eating. The quality of life is consequently significantly reduced in the affected patients.

Oral mucositis (stomatitis) with ulcers can manifest itself already a few days after onset of chemotherapy and/or radiotherapy, and is precipitated by a direct toxic effect on the mucosal membranes. The direct toxic effect is caused by nonspecific killing of rapidly dividing basal epithelial cells resulting in epithelial thinning, inflammation, decreased cell renewal, and ultimately ulceration.

Postoperative radiotherapy is usually indicated in patients after resection of advanced carcinomas of the head and neck. Recently two randomized phase III studies performed by the European Organization for Research and Treatment of Cancer (EORTC) and an intergroup effort of Radiation Therapy Oncology Group (RTOG), Eastern Cooperative Oncology Group (ECOG) and Southwest Oncology Group (SWOG) demonstrated that adjunct cisplatin-based chemotherapy significantly improves the local and regional tumor control compared to radiotherapy alone. The disease-free survival was also significantly longer in both studies. However, this improvement in efficacy was accompanied by a higher incidence of acute adverse events of grade 3 or greater, especially oral mucositis, in the group receiving combined treatment. Despite of increased toxicity, the authors conclude that the addition of concurrent chemotherapy to radiotherapy will be the new standard of care for physically fit patients with head and neck cancer (Bernier et al. 2004; Cooper et al. 2004).

Aside from the direct effect of radiation or chemotherapy on mucosal cells, mucositis might also be caused by therapy induced neutropenia. Drugs that commonly give dose-limiting oral mucositis are: methotrexate, dactinomycin, and doxorubicin, but also bleomycin, cytarabin, fluorouracil, and mitramycin induce this side effect. The side effect is dose- and schedule related. The combination of cisplatin and continuous infusion of fluorouracil for squamous cell head and neck carcinoma almost always results in severe mucositis (Sonis et al. 1990).

Patients undergoing chemotherapy often suffer from myelosuppression due to their treatment, and this might induce indirect mucositis. Severe granulocytopenia is conductive to oral infections by Gram-negative bacilli, Gram-positive cocci, fungi such as Candida species, and viruses (particularly Herpes simplex). These infections usually occur at the site of direct mucositis or other oral trauma 12-14 days after drug administration (Sonis et al. 1990; Verdi 1993).

Atrophic changes are usually seen after a dose of about 2000 cGy administered at a rate of 200 cGy a day. Radiation following chemotherapy may lead to especially severe complications. The degree of damage is directly related to the dose of radiation, but also to factors as age, concurrent diseases (e.g. AIDS), oral hygiene, and tobacco and alcohol usage. Previous treatment may also affect the outcome. The general nutritional and health status of the patient also plays an important role in determining the severity of the complications and both of these can be adversely affected by the complications themselves creating a vicious circle (Reynolds et al. 1980; Shannon et al. 1977; Baker 1982). If the complications are sufficiently severe they will lead to an interruption of treatment and thus, possibly, a complete or partial failure of therapy.

The previous notion that radiation injury of the oral mucosa is a purely epithelial phenomenon has been supplanted by the recognition that it, similar to radiation injury in other organ systems, is a dynamic process of complex interactions among many cellular compartments, resulting in a number of concurrent and sequential pathophysiological alterations that collectively constitute what is called radiation-induced stomatitis or oral mucositis.

Oral mucositis is heralded by an initial phase that is characterized by injury to tissues of the submucosa. During this phase, changes are mediated by reactive oxygen species (ROS) through the ceramide pathway, and by activation of a number of transcription factors including nuclear factor-kappa beta (NF-κB). This results in the activation of early response genes, as well as direct oxidative alterations of protein functions, such as, proteins responsible for vascular thromboresistance. Changes occur in endothelial cells, mesenchymal cells, resident inflammatory cells, and extracellular matrix. The initial injury precipitates the upregulation of a second set of genes resulting in direct and indirect signaling and early apoptosis of clonogenic stem cells in the basal epithelium. The signaling molecules are likely to be the proinflammatory cytokines (tumor necrosis factor-α, interleukin 1, and interleukin 6). These signaling molecules also have the ability to further amplify the upregulation of transcription factors (e.g., NF-κB), leading to production of additional proinflammatory cytokines, tissue injury, and apoptosis.

Deficient renewal of mucosal epithelium occurs despite focal bursts of hyperproliferative activity in response to upregulation of genes associated with epithelium healing. When epithelial apoptosis and necrosis exceeds hyperproliferative activity, an ulcerative phase with full thickness mucosal damage is the visible result. The ulcerative phase is exacerbated by local bacterial colonization, which results in a barrage of cell wall products that penetrates into the submucosa and amplifies the damage.

Eventually, healing occurs as healthy epithelium migrates from the wound margins, stimulated by signals from the submucosa, and cytokines and other mediators drive additional local response, including angiogenesis.

Management of Mucositis

Mucositis and ulceration in the oral cavity can be extremely painful and are a major site of potentially lethal infections. Although many phase I and II studies have been performed with products having shown promising results in animal models (see Velez et al. 2004. Quintessence Int. vol 35:129-136, Management or oral mucositis induced by chemotherapy and radiotherapy: an update), very few, if any, treatments have been demonstrated to be effective in preventing or treating oral mucositis.

Several agents have been investigated in order to find optimal management principles for mucositis and even though some agents have shown prophylactic effect (Meisenberg et al., 1996), no agent has been shown to be efficient in all settings. Treatment of mucositis is today primarily supportive; strong analgesics in addition to oral hygiene. No standard therapy has been accepted.

Several authors have reviewed the literature on treatment and prevention strategies for chemotherapy- and radiotherapy-induced oral mucositis, and conclude that further trials are needed (Clarkson et al. 2000; Worthington and Clarkson 2002; Sonis et al. 2004).

Both topical and systemic prophylactic agents as well as non-pharmacologic prophylaxis are among the treatment regimens that have been tried in oral mucositis. Some cancer treatment regimens make use of specific antidotes (e.g. leucovorin) after moderate-dose or high-dose methotrexate (Allegra and Boarman 1990) to reduce the toxic effect of the cancer drug. Due to the mechanism of action of antiseptics and antifungals, prophylactic chlorhexidine (Ferretti et al. 1990; McGaw and Belch 1985) and nystatin or clotrimazole (Preston and Briceland 1995) reduce the risk of indirect mucotoxicity from bacteria and fungi. Sucralfate is a basic aluminum salt of sulfated sucrose which forms an ionic bond to proteins in ulcerations. This produces a protective barrier that promotes healing. Sucralfate has shown a modest benefit in patients receiving a cisplatin/fluorouracil regimen for various solid tumors (Pfeiffer et al. 1990). Oral cryotherapy/ice chips have been shown to have effect in patients receiving bolus doses of 5-fluorouracil (Rocke et al. 1993), but unsuitable for patients receiving continuous infusion of 5-fluorouracil.

Interventions that have failed to show effect in oral mucositis, include the application of prostaglandin E2 (Labar et al. 1993), to some extent allopurinol (xanthine oxidase inhibitor) (Loprinzi et al. 1995), pentoxifylline (PTX) (Stockschlader et al. 1993; Attal et al. 1993; van der Jagt et al. 1994) and filgrastim, a granulocyte colony-stimulating factor (Gabrilove et al. 1988; Pettengell et al. 1992).

β-glucans in Radioprotection

“β-glucan” is the common name for homopolysaccharides consisting of β-D-glucopyranosyl units. The backbone units are linked by β-1,3- or β-1,4-linkages, or combinations of these two. β-glucans from different sources and isolated through different methods may have a variety of additional structural features like single glucosyl units attached to the backbone, β-1,6-linked side chains or β-1,3-linked side chains at different ratios.

It has been known for many years that injection of β-glucan to animals receiving radiation can induce enhanced hematopoietic recovery (Patchen et al. 1984) and increased survival rate (Patchen and MacVittie 1986; Hofer et al. 1995) from cobalt-60 radiation. The increase in survival rate is hypothesized to be due to the prevention of radiation induced myelosuppression and stimulation of the bone marrow (Patchen et al. 1990; Hofer and Pospisil 1997), a theory that has been supported by recent findings that yeast β-glucan is able to induce increased bone marrow mononuclear cell colony formation (Turnbull et al. 1999). All the above studies refer to parenteral administration of β-glucans. No studies have earlier been carried out to examine the effect of orally administered β-glucan on radio- or chemotherapy induced side-effects such as mucositis.

In the present invention it was surprisingly found that water-soluble β-1,3-glucan is effective in preventing or treating oral mucositis and ulceration in cancer patients undergoing radiation treatments to head and neck. Accordingly, novel methods of prevention and treatment, novel use of β-glucans, as well as novel use of β-glucans for manufacturing a medicament for preventing or treating mucositis are devised herein.

### SUMMARY OF THE INVENTION

The current invention describes a method for preventing and/or treating mucositis with a branched water soluble β-1,3-glucan. Water soluble β-glucan is used to prevent development of and/or promote healing of ulcers as exemplified by mouth and oropharynx ulcerations resulting from radio- and/or chemotherapy. The soluble β-glucan can be applied directly to the potential affected mucosal areas as a solution, or mixture, or rinse, or gel, or mixed into any pharmaceutically acceptable carrier or vehicle. Other aspects of the invention are glucans for use in therapeutic compositions and uses of β-glucan.

### DETAILED DESCRIPTION OF THE INVENTION

The present invention relates to prevention or treatment of mucositis. More particularly, it relates to a method of preventing or treating oral mucositis. In a preferred embodiment, the oral mucositis is radiation and/or chemotherapy induced oral mucositis. The method of the invention comprises applying a preparation comprising water-soluble immunomodulatory β-glucan to the susceptible or affected mucosal surface. The preparation may be a rinse, mixture, gel, ointment, cream, or another suitable formulation. Furthermore, water-soluble β-glucan may be the only active component in the preparation or the β-glucan may be combined with one or several other active components like e.g. antiseptics and/or antifungals, e.g. chlorhexidine, nystatin, clotrimazole; sucralfate; analgesics; etc.

The water-soluble immunomodulatory β-glucan used in the invention may be isolated from several organisms. Glucans with known immunomodulatory activities are e.g. lentinan isolated from Lentinus edodes having a β-1,6-linked single glucosyl unit for approximately every 3 main chain unit (Sasaki and Takasuka 1976). Similar glucans having β-1,3-linked main chain with single β-1-6-linked glucosyl units attached thereto are scleroglucan isolated from Sclerotium sp. (Singh et al. 1974; Farina et al. 2001) and shizophyllan isolated from Schizophyllum commune (Akima et al. 1985). A soluble β-1,3-glucan can also be obtained from seaweed (Nelson and Lewis 1974), and a water soluble β-1,3/1,4-glucan can be isolated from cereals (Estrada et al. 1997), or derived from lichens (Demleitner et al. 1992). Bacterial β-1,3-glucan, curdlan, from Alcaligenes faecalis can be made water soluble and immunomodulatory (Seljelid et al. 1984). Preferably, the β-glucan originates from yeast, fungi, cereals, algae, or bacteria. More preferably, said glucan originates from yeast. Even more preferably, said glucan originates from the yeast family Saccharomyces. In a particularly preferred embodiment, the β-glucan originates from Saccharomyces cerevisiae. The water-soluble immunomodulatory β-glucan may be of any structure, however, it is preferably a water-soluble non-derivatized β-1,3-glucan, with side-chains anchored to the backbone through a β-1,6-linkage. Preferably, the backbone consists of β-1,3-linked D-glucopyranosyl units, while the side chains may comprise β-1,3-linked and/or β-1,6-linked D-glucopyranosyl units. The former type of side-chains are termed β-1,3 side-chains, while the latter are termed β-1,6 side-chains. Preferably, said β-1,6 side-chains consist of 0 to 4 units. More preferably, said side-chains consist exclusively of β-1,3-linked D-glucopyranosyl units. The above features are important both for the current β-glucan's water solubility and immunomodulatory activity (Engstad 1994). As teached above, the glucan may contain certain amounts of β-1,6-linked glucosyl chains, but these are to a certain extent negative with respect to the β-glucans immunomodulatory abilities (see Engstad 1994), the content of which are incorporated herein by reference. These undesired β-1,6 side-chains are found in other non-derivatized soluble yeast β-glucans described in the literature (Onderdonk et al. 1992) or patents (Jamas et al. 1994; Kelly 2001). Accordingly, an especially preferred glucan is a branched β-1,3-glucan with β-1,3 side chains anchored through a β-1,6-linkage.

The β-glucan concentration of the preparation may be in the range from 0.1% to 25% by weight, preferably 0.1% to 10% by weight, more preferably 0.5% to 2.5% by weight. The preparation may be administered either as a single daily treatment or repeated daily treatments before, and/or under, and/or after e.g. a cancer treatment regime or bone marrow transplantation. The preparation may be administered orally to contact the mucosal surfaces of the oral cavity, the pharynx and the intestinal tract to prevent and/or heal the formation of mucositis, e.g. in radio- or chemotherapy treated cancer patients. Anal administration of the preparation is also possible. The method of the present invention may be applied to any animal, preferably a mammal, and more preferably a human being.

Another aspect of the current invention is a method for preparing a medicament comprising the β-glucan as described above. The process comprises first isolating intact yeast cell walls or any other source of β-glucan as described above. The intact cell walls, or alternative source, are treated with formic acid and optionally digested with a β-(1,6)-glucanase to form a gel with non-Newtonian viscosity and thixotropic properties. This type of gel is ideal for mucosal application. A general method for isolation and manufacture of the current β-glucan is described in patent EP 0759089 the content of which are incorporated herein by reference.

A further aspect of the current invention is a glucan for use in a therapeutic composition for treatment or prevention of mucositis, wherein said glucan is a water-soluble immunomodulatory β-1,3-glucan. The β-glucan is as described above. Preferably, the water-soluble immunomodulatory β-1,3-glucan have a branched nature with β-1,3-linked side chains anchored through a β-1,6-linkage. More preferably, said side-chains consist exclusively of β-1,3-linked D-glucopyranosyl units. In a preferred embodiment the mucositis is oral mucositis. The mucositis may be caused by radiotherapy and/or chemotherapy.

Yet another aspect of the current invention is the use of immunomodulatory β-glucan for manufacturing a medicament for the treatment or prevention of mucositis in an animal in need thereof. The β-glucan is as described above. Preferably, the water-soluble immunomodulatory β-1,3-glucan is branched with β-1,3-linked side chains anchored through a β-1,6-linkage. More preferably, said side-chains consist exclusively of β-1,3-linked D-glucopyranosyl units. In a preferred embodiment the mucositis is oral mucositis. The mucositis may be caused by radiotherapy and/or chemotherapy. The animal may be any animal, preferably a mammal, and most preferably a human being.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

The theory underlying the invention is not part of the claims and the inventor does not wish to be bound by any particular theory explaining the invention. In fact, it is fully anticipated that the theory underlying the present invention will evolve as science develop and mature.

### EXAMPLES

The following examples are meant to illustrate how to make and use the invention. They are not intended to limit the scope of the invention in any manner or to any degree.

### Example I

Herein is described the use of soluble β-glucan (SBG) to prevent and or heal oral mucositis in conjunction to radiotherapy in a 30 year old male with head and neck cancer. SBG is a water soluble branched beta-1,3-linked yeast derived glucan with intact beta-1,3-linked side chains anchored through a beta-1,6-linkage to the main chain. A minority of side chains show repetitive beta-1,6-linkages. The intramolecular ratio of repetitive beta-1,6-linkages versus beta-1,3-linkages is approximately 1:50, or less. The patient was diagnosed with cancer in the tongue in January 2002 and a surgical excision of the tumour was performed, whereafter local brachy therapy in the tongue was given. In November 2002, 10 months after surgery, recurrence of the cancer was diagnosed in a lymph node in the neck, and the patient was readmitted to hospital for surgery in December 2002 and from January 2003 also radiotherapy to the neck region. Radiotherapy (a total of 60 Gy) was given 5 days per week for 6 consecutive weeks. Concomitant to the radiotherapy the patient took approximately 80-100 mg SBG as a 20 mg/ml aqueous solution administered orally as a daily dose until 14 days after ending radiotherapy. The patient did not develop oral mucositis above grade I during or after the radiotherapy.

### Example II

An exploratory, randomized, parallel group study comparing the protective effect of soluble β-glucan or placebo in oral mucositis in head and neck cancer patients receiving radiation therapy is described. 40 patients undergoing radiation for histologically confirmed squamous cell carcinoma of the oral cavity or pharynx (1.8-2.0 Gy/day, 5 days per week; totally 59.4-70 Gy) is included in the study. A cohort also receives chemotherapy. Soluble β-glucan (SBG) as an aqueous solution is given orally throughout the whole radiation period at a daily dosage of 500-1000 mg as a 15 mg/ml aqueous solution to 20 patients, whereas 20 patients are treated with methylcellulose as placebo. SBG is a water soluble branched beta-1,3-linked yeast derived glucan with intact beta-1,3-linked side chains anchored through a beta-1,6-linkage to the main chain. A minority of side chains show repetitive beta-1,6-linkages. The intramolecular ratio of repetitive beta-1,6-linkages versus beta-1,3-linkages is approximately 1:50, or less.

The β-glucan treatment group shows reduction in number of patient having grade 2 or higher oral mucositis as compared to the placebo group.

Having now fully described the present invention in some detail by way of illustration and example for purpose of clarity of understanding, it will be obvious to one of ordinary skill in the art that same can be performed by modifying or changing the invention by with a wide and equivalent range of conditions, formulations and other parameters thereof, and that such modifications or changes are intended to be encompassed within the scope of the appended claims.

### REFERENCE LIST

Akima, K., Arika, T., and Amemiya, K. 1985. Purification, physicochemical characterization, and antitumor activity of a cancer-associated human serum protein that is increased by treatment with schizophyllan, an antitumor polysaccharide. Japanese Journal of Cancer Research (Gann) 76, 541-48.
Allegra, C. J. and Boarman, D. 1990. Interaction of methotrexate polyglutamates and dihydrofolate during leucovorin rescue in a human breast cancer cell line (MCF-7). Cancer Res 50, 3574-3578.
Attal, M., Huguet, F., Rubie, H., Charlet, J. P., Schlaifer, D., Huynh, A., Laurent, G., and Pris, J. 1993. Prevention of regimen-related toxicities after bone marrow transplantation by pentoxifylline: a prospective, randomized trial. Blood 82, 732-736.
Baker, D. G. 1982. The radiobiological basis for tissue reactions in the oral cavity following therapeutic x-irradiation. A review. Arch. Otolaryngol. 108, 21-24.
Bernier, J., Domenge, C., Ozsahin, M., Matuszewska, K., Lefebvre, J. L., Greiner, R. H., Giralt, J., Maingon, P., Rolland, F., Bolla, M., Cognetti, F., Bourhis, J., Kirkpatrick, A., and van Glabbeke, M. 2004. Postoperative irradiation with or without concomitant chemotherapy for locally advanced head and neck cancer. N. Engl. J Med 350, 1945-1952.
Clarkson, J. E., Worthington, H. V., and Eden, O. B. 2000. Prevention of oral mucositis or oral candidiasis for patients with cancer receiving chemotherapy (excluding head and neck cancer). Cochrane. Database. Syst. Rev CD000978.
Cooper, J. S., Pajak, T. F., Forastiere, A. A., Jacobs, J., Campbell, B. H., Saxman, S. B., Kish, J. A., Kim, H. E., Cmelak, A. J., Rotman, M., Machtay, M., Ensley, J. F., Chao, K. S., Schultz, C. J., Lee, N., and Fu, K. K. 2004. Postoperative concurrent radiotherapy and chemotherapy for high-risk squamous-cell carcinoma of the head and neck. N. Engl. J Med 350, 1937-1944.
Demleitner, S., Kraus, J., and Franz, G. 1992. Synthesis and antitumor activity of sulfoalkyl derivatives of curdlan and lichenan. Carbohydrate Research 226, 247-252.
Dodd, M. J., Facione, N. C., Dibble, S. L., and MacPhail, L. 1996. Comparison of methods to determine the prevalence and nature of oral mucositis. Cancer Pract. 4, 312-318.
Engstad, R. E. 1994. Yeast β-glucan as an immunostimulant in Atlantic salmon (Salmo salar L.): Biological effects, recognition and structural aspects., University of Tromsø, Tromsø.
Estrada, A., Yun, C. H., van Kessel, A., Li, B., Hauta, S., and Laarveld, B. 1997. Immunomodulatory activities of oat beta-glucan in vitro and in vivo 2. Microbiol. Immunol. 41, 991-998.
Farina, J. I., Sineriz, F., Molina, O. E., and Perotti, N. I. 2001. Isolation and physicochemical characterization of solublescleroglucan from Sclerotium rolfsii. Rheological properties, molecular weight and conformational characteristics. Carbohydrate Polymers 44, 41-50.
Ferretti, G. A., Brown, A. T., Raybould, T. P., and Lillich, T. T. 1990. Oral antimicrobial agents—chlorhexidine. NCI. Monogr 51-55.
Gabrilove, J. L., Jakubowski, A., Scher, H., Sternberg, C., Wong, G., Grous, J., Yagoda, A., Fain, K., Moore, M. A., Clarkson, B., and . 1988. Effect of granulocyte colony-stimulating factor on neutropenia and associated morbidity due to chemotherapy for transitional-cell carcinoma of the urothelium. N. Engl. J Med 318, 1414-1422.
Hofer, M. and Pospisil, M. 1997. Glucan as stimulator of hematopoiesis in normal and gamma-irradiated mice. A survey of authors results. International Journal of Immunopharmacology 19, 607-609.
Hofer, M., Pospisil, M., Viklicka, S., Pipalova, I., Hola, J., Netikova, J., and Sandula, J. 1995. Effects of postirradiation carboxymethylglucan administration in mice. Int. J. Immunopharmacol. 17, 167-174.
Jamas, S., Easson, D. D., and Ostroff, G. R. Method for producing neutral glucans for pharmaceutical applications. U.S. Pat. No. 5,322,841.
Kelly, G. E. Process for glucan preparation and therapeutic uses of glucan. U.S. Pat. No. 6,242,594.
Labar, B., Mrsic, M., Pavletic, Z., Bogdanic, V., Nemet, D., Aurer, I., Radman, I., Filipovic-Grcic, N., Sertic, D., Kalenic, S., and 1993. Prostaglandin E2 for prophylaxis of oral mucositis following BMT. Bone Marrow Transplant. 11, 379-382.
Loprinzi, C. L., Foote, R. L., and Michalak, J. 1995. Alleviation of cytotoxic therapy-induced normal tissue damage. Semin. Oncol. 22, 95-97.
McGaw, W. T. and Belch, A. 1985. Oral complications of acute leukemia: prophylactic impact of a chlorhexidine mouth rinse regimen. Oral Surg. Oral Med Oral Pathol. 60, 275-280.
Nelson, T. E. and Lewis, B. A. 1974. Separation and characterization of the soluble and insoluble components of insoluble laminaran. Carbohydrate Research 33, 63-74.
Onderdonk, A. B., Cisneros, R. L., Hinkson, P., and Ostroff, G. 1992. Anti-infective effect of poly-β1-6-Glucotriosyl-β1-3-glucopyranose glucan in vivo. Infection and Immunity 60, 1642-1647.
Patchen, M. L., Di Luzio, N. R., Jacques, P., and MacVittie, T. J. 1984. Soluble polyglucans enhance recovery from cobalt-60-induced hemopoietic injury. Journal of Biological Response Modifiers 3, 627-633.
Patchen, M. L. and MacVittie, T. J. 1986. Comparative effects of soluble and particulate glucans on survival in irradiated mice. Journal of Biological Response Modifiers 5, 45-60.
Patchen, M. L., MacVittie, T. J., Solberg, B. D., and Souza, L. M. 1990. Survival enhancement and hemopoietic regeneration following radiation exposure: therapeutic approach using glucan and granulocyte colony-stimulating factor. Exp. Hematol. 18, 1042-1048.
Pettengell, R., Gurney, H., Radford, J. A., Deakin, D. P., James, R., Wilkinson, P. M., Kane, K., Bentley, J., and Crowther, D. 1992. Granulocyte colony-stimulating factor to prevent dose-limiting neutropenia in non-Hodgkin's lymphoma: a randomized controlled trial. Blood 80, 1430-1436.
Pfeiffer, P., Madsen, E. L., Hansen, O., and May, O. 1990. Effect of prophylactic sucralfate suspension on stomatitis induced by cancer chemotherapy. A randomized, double-blind cross-over study. Acta Oncol. 29, 171-173.
Preston, S. L. and Briceland, L. L. 1995. Fluconazole for antifungal prophylaxis in chemotherapy-induced neutropenia. Am. J Health Syst. Pharm. 52, 164-173.
Reynolds, W., Hickey, A., and Feldman, M. 1980. Dental management of the cancer patient receiving radiation therapy. Clin. Prevent. Dent 2, 5-9.
Rocke, L. K., Loprinzi, C. L., Lee, J. K., Kunselman, S. J., Iverson, R. K., Finck, G., Lifsey, D., Glaw, K. C., Stevens, B. A., Hatfield, A. K., and 1993. A randomized clinical trial of two different durations of oral cryotherapy for prevention of 5-fluorouracil-related stomatitis. Cancer 72, 2234-2238.
Sasaki, T. and Takasuka, N. 1976. Further study of the structure of lentinan, an anti-tumor polysaccharide from Lentinus edodes. Carbohydrate Research 47, 99-104.
Seljelid, R., Bogwald, J., Hoffman, J., and Larm, O. 1984. A soluble β-1,3-D-glucan derivative potentiates the cytostatic and cytolytic capacity of mouse peritoneal macrophages in vitro. Immunopharmacology 7, 69-73.
Shannon, I. L., Starcke, E. N., and Wescott, W. B. 1977. Effect of radiotherapy on whole saliva flow. J Dent Res 56, 693.
Singh, P. P., Whistler, R. L., Tokuzen, R., and Nakahara, W. 1974. Scleroglucan, an antitumor polysaccharide from Sclerotium glucanicum. Carbohydr. Res 37, 245-247.
Sonis, S. T., Elting, L. S., Keefe, D., Peterson, D. E., Schubert, M., Hauer-Jensen, M., Bekele, B. N., Raber-Durlacher, J., Donnelly, J. P., and Rubenstein, E. B. 2004. Perspectives on cancer therapy-induced mucosal injury: pathogenesis, measurement, epidemiology, and consequences for patients. Cancer 100, 1995-2025.
Sonis, S. T., Woods, P. D., and White, B. A. 1990. Oral complications of cancer therapies. Pretreatment oral assessment. NCI. Monogr 29-32.
Stockschlader, M., Kalhs, P., Peters, S., Zeller, W., Kruger, W., Kabisch, H., Lechner, K., and Zander, A. 1993. Intravenous pentoxifylline failed to prevent transplant-related toxicities in allogeneic bone marrow transplant recipients. Bone Marrow Transplant. 12, 357-362.
Trotti, A. 2000. Toxicity in head and neck cancer: a review of trends and issues. Int. J Radiat. Oncol. Biol Phys. 47, 1-12.
Turnbull, J. L., Patchen, M., and Scadden, D. T. 1999. The polysaccharide, PGG-glucan, enhances human myelopoiesis by direct action independent of and additive to early-acting cytokines. Acta Haematol. 102, 66-71.
van der Jagt, R. H., Pari, G., McDiarmid, S. A., Boisvert, D. M., and Huebsch, L. B. 1994. Effect of pentoxifylline on regimen related toxicity in patients undergoing allogeneic or autologous bone marrow transplantation. Bone Marrow Transplant. 13, 203-207.
Verdi, C. J. 1993. Cancer therapy and oral mucositis. An appraisal of drug prophylaxis. Drug Saf 9, 185-195.
Worthington, H. V. and Clarkson, J. E. 2002. Prevention of oral mucositis and oral candidiasis for patients with cancer treated with chemotherapy: cochrane systematic review. J Dent Educ. 66, 903-911.

What is claimed is:

1. A method of treating and/or preventing mucositis comprising applying to the mucosal surface a preparation comprising water-soluble immunomodulatory β-glucan

2. The method of claim 1, wherein the glucan is isolated from yeast, fungi, cereals, algae, or bacteria.

3. The method of claim 1, wherein the glucan is isolated from yeast.

4. The method of claim 3, wherein the yeast is Saccharomyces cerevisiae.

5. The method of claim 1, wherein the glucan is a branched β-1,3-glucan with β-1,3 side chains anchored through a β-1,6-linkage.

6. The method of claim 5, wherein the side chains exclusively consist of β-1,3 linked D-glucopyranosyl units.

7. The method according to claim 1, wherein the glucans are used in amounts of 0.1% to 25% by weight based on the preparation.

8. The method of claim 1, wherein the mucositis is oral mucositis.

9. A glucan for use in a therapeutic composition for treatment or prevention of mucositis, wherein said glucan is a water-soluble immunomodulatory β-1,3-glucan having a branched nature with β-1,3-linked side chains anchored through a β-1,6-linkage.

10. The glucan of claim 9, wherein the glucan is isolated from yeast.

11. The glucan of claim 10, wherein the yeast is Saccharomyces cerevisiae.

12. The glucan of claim 9, wherein the side chains exclusively consist of β-1,3 linked D-glucopyranosyl units.

13. The glucan of claim 9, wherein the mucositis is oral mucositis.

14. Use of immunomodulatory β-glucan for manufacturing a medicament for the treatment or prevention of mucositis in an animal in need thereof.

15. Use of claim 14, wherein the glucan is isolated from yeast.

16. Use of claim 15, wherein the yeast is Saccharomyces cerevisiae.

17. Use of claim 14, wherein the β-glucan is a β-1,3-glucan having a branched nature with β-1,3-linked side chains anchored through a β-1,6-linkage.

18. The use of claim 17, wherein the side chains exclusively consist of β-1,3 linked D-glucopyranosyl units.

19. Use of claim 14, wherein the mucositis is oral mucositis.

20. Use of claim 14, wherein the animal is a mammal.

21. Use of claim 20, wherein the mammal is a human being.
